(19) United States
(12) Patent Application Publication
(10) Pub. No.: US 2024/0328553 A1
(43) Pub. Date: Oct. 3, 2024

(54) CONNECTOR COUPLING ASSEMBLY

(71) Applicant: CareFusion 303, Inc., San Diego, CA (US)

(72) Inventors: Abin AUSTIN, Thrissur (IN); Mohammed Mehtab KHAN, Bengaluru (IN); Aman DESAI, Bengaluru (IN)

(21) Appl. No.: 18/595,972

(22) Filed: Mar. 5, 2024

Related U.S. Application Data
(60) Provisional application No. 63/455,206, filed on Mar. 28, 2023.

(51) Int. Cl. F16L 37/35; A61M 39/26

(52) U.S. Cl. CPC F16L 37/35; A61M 39/26; A61M 2039/263

(57) ABSTRACT

A coupler including a first connector having a first end, a second end opposite the first end, a first opening, and a first valve disposed between the first end and the second end, the first valve having a compressed and an expanded state, the first valve extending through the first opening when the first valve is in the expanded state and a second connector having a coupling portion, a second opening, and a second valve disposed at least partially within the coupling portion. The second valve having a compressed and an expanded state, the second valve extending at least partially through the second opening when the second valve is in the expanded state. The first valve and the second valve are in the compressed state when the first connector is coupled to the second connector such that a fluid pathway is formed through the first connector and the second connector.

### CROSS-REFERENCE TO RELATED APPLICATION

This application claims the benefit of U.S. Provisional Patent Application No. 63/455,206 filed Mar. 28, 2023, entitled “Connector Coupling Assembly”, which is incorporated by reference herein in its entirety.

### FIELD OF THE INVENTION

The present disclosure generally relates to connectors, and, in particular, to connector couplings.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. Often, tubing or catheters are coupled or secured to each other to allow fluid communication between various portions of tubing or catheters.

In some applications, such tubing or catheters may become dislodged due to improper securement and/or when the coupling is subject to forces greater than what the coupling is designed to withstand.

### SUMMARY

One or embodiments of the present disclosure are directed to a coupler including a first connector having a first end, a second end opposite the first end, a first opening, and a first valve disposed between the first end and the second end, the first valve having a compressed state and an expanded state, the first valve extending at least partially through the first opening when the first valve is in the expanded state and a second connector having a coupling portion, a second opening, and a second valve disposed at least partially within the coupling portion, the second valve having a compressed state and an expanded state, the second valve extending at least partially through the second opening when the second valve is in the expanded state. The first connector is configured to receive at least a portion of the second connector to detachably couple the first connector to the second connector such that the coupling portion of the second connector extends through the second end of the first connector, and the first valve and the second valve are in the compressed state when the first connector is coupled to the second connector such that a fluid pathway is formed through the first connector and the second connector,

In some embodiments, the first connector is configured to decouple from the second connector in response to a pullout force exceeding a predetermined threshold force.

In some embodiments, the first valve seals the first opening when the first valve is in the expanded state. In some embodiments, the second valve seals the second opening when the second valve is in the expanded state.

In some embodiments, the first connector includes a first biasing element coupled to the first valve, the first biasing element having an expanded state and a compressed state. The first biasing element being in the expanded state results in the first valve being in the expanded state and the first biasing element being in the compressed state results in the first valve being in the compressed state.

In some embodiments, the second connector includes a second biasing element coupled to the second valve, the second biasing element having an expanded state and a compressed state. The second biasing element being in the expanded state results in the second valve being in the expanded state and the second biasing element being in the compressed state results in the second valve being in the compressed state.

In some embodiments, the pullout force is a force applied to the first connector along a central axis of the first connector and the central axis extends at least along a length of the first connector. The central axis extends through the first connector and the second connector when the first connector is coupled to the second connector.

In some embodiments, the first valve includes a first substantially planar surface and the second valve includes a second substantially planar surface, the first substantially planar surface configured to contact the second substantially planar surface when the first connector is coupled to the second connector.

In some embodiments, the first valve exerts a pressure on the second valve causing the second valve to be in the compressed state when the first connector is coupled to the second connector.

In some embodiments, the first valve at least partially extends through the first opening to seal the first opening when the first connector is decoupled from the second connector.

In some embodiments, the second valve at least partially extends through the second opening to seal the second opening when the first connector is decoupled from the second connector.

In some embodiments, the first connector includes a mating portion configured to receive the coupling portion of the second connector when the first connector is coupled to the second connector.

In some embodiments, the second valve is in the expanded state when the first connector is disconnected from the second connector. The first valve is in the expanded state when the first connector is disconnected from the second connector.

In some embodiments, the first connector is configured to remain coupled to the second connector when the pullout force does not exceed the predetermined threshold force.

In some embodiments, the first valve does not overlap with the second valve. The first valve abuts the second valve when the first connector is coupled to second connector.

In some embodiments, the coupler has a first configuration and in the first configuration the first connector is coupled to the second connector such that the second connector is at least partially disposed within the first connector.

In some embodiments, the coupler has a second configuration and in the second configuration the first connector is disconnected from the second connector.

In some embodiments, the first connector is coupled to a first portion of tubing at the first end and the second connector is coupled to a second portion of tubing at an output portion.

One or more embodiments of the present disclosure are directed to a coupler including a first connector having a first end, a second end opposite the first end, a mating portion disposed proximate the second end, a first opening disposed between the first end and the second end, a biasing element disposed within the first connector, and a first valve coupled to the biasing element and disposed between the first end and the first opening, the first valve and the first biasing element having a compressed state and an expanded state, the first biasing element being in the expanded state causes the first valve to be in the expanded state resulting in the first valve at least partially extending through the first opening to seal the first opening, and a second connector having a coupling portion, a biasing element disposed within the second connector, a second opening, and a second valve coupled to the biasing element and disposed at least partially within the coupling portion, the second valve and the second biasing element having a compressed state and an expanded state, the second biasing element being in the expanded state causes the second valve to be in the expanded state resulting in the second valve at least partially extending through the second opening to seal the second opening. The mating portion of first connector is configured to receive the coupling portion of the second connector to detachably couple the first connector to the second connector such that the coupling portion of the second connector extends through the second end of the first connector, and the first biasing element being in the compressed state causes the first valve to be in the compressed state and the second biasing element being in the compressed state causes the second valve to be in the compressed state such that a fluid pathway is formed through the first connector and the second connector via the first opening and the second opening. The first connector is configured to decouple from the second connector in response to a pullout force exceeding a predetermined threshold force.

One or more embodiments of the present disclosure are directed to a coupler including a first connector having a first end, a second end opposite the first end, an inlet portion disposed proximate the first end, a mating portion in fluid communication with the inlet portion and disposed proximate the second end, a first opening disposed between the first end and the second end, a first interior space disposed between the first end and the first opening, a biasing element disposed within the interior space, and a first valve coupled to the biasing element and disposed between the first end and the first opening, the first valve and the first biasing element having a compressed state and an expanded state, the first biasing element being in the expanded state causes the first valve to be in the expanded state resulting in the first valve at least partially extending through the first opening to seal the first opening, and a second connector having a coupling portion, an output portion extending from the coupling portion, an interior space disposed within the coupling portion, a biasing element disposed within the interior space, a second opening, and a second valve coupled to the biasing element and disposed at least partially within the coupling portion, the second valve and the second biasing element having a compressed state and an expanded state, the second biasing element being in the expanded state causes the second valve to be in the expanded state resulting in the second valve at least partially extending through the second opening to seal the second opening. The mating portion of first connector is configured to receive the coupling portion of the second connector to detachably couple the first connector to the second connector such that the coupling portion of the second connector extends through the second end of the first connector, and the first biasing element being in the compressed state causes the first valve to be in the compressed state and the second biasing element being in the compressed state causes the second valve to be in the compressed state such that a fluid pathway is formed through the first connector and the second connector via the first opening and the second opening. The first valve includes a first substantially planar surface, and the second valve includes a second substantially planar surface, the first substantially planar surface configured to contact the second substantially planar surface when the first connector is coupled to the second connector. The first connector is configured to decouple from the second connector in response to a pullout force exceeding a predetermined threshold force, the pullout force being a force applied to the first connector along a central axis of the first connector and the central axis extends at least along a length of the first connector.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a system diagram showing a coupler assembly in use, in accordance with various aspects of the present disclosure.

FIG. 2 is a cross-sectional side view of the coupler assembly of FIG. 1, in accordance with various aspects of the present disclosure.

FIG. 3 is a cross-sectional side view of a first connector of the coupler assembly of FIG. 1, in accordance with various aspects of the present disclosure.

FIG. 4 is a cross-sectional side view of a second connector of the coupler assembly of FIG. 1, in accordance with various aspects of the present disclosure.

FIG. 5 is a cross-sectional side view of the coupler assembly of FIG. 1 with the first connector coupled to the second connector, in accordance with various aspects of the present disclosure.

FIG. 6 is a cross-sectional side view of the coupler assembly of FIG. 1 with the first connector decoupled from the second connector, in accordance with various aspects of the present disclosure.

### DETAILED DESCRIPTION

The disclosed coupler assembly includes a first connector and a second connector. The first connector is configured to couple to the second connector. The coupler assembly may have a first configuration and a second configuration. In the first configuration, the first connector is coupled to the second connector. In the second configuration, the first connector is decoupled from the second connector.

The coupler assembly may be configured to couple a first portion of tubing to a second portion of tubing. For example, the first portion of tubing may be coupled to the first connector and the second portion of tubing may be coupled to the second connector. The first portion of tubing and/or the second portion of tubing may also couple to a patient or fluid source. In some embodiments, the coupler assembly allows for the flow of fluid from the first portion of tubing to the second portion of tubing. For example, the first connector may be coupled to the second connector such that a fluid pathway is formed through the first connector and the second connector to allow the flow of fluid from the first portion of tubing through the first connector and the second connector to the second portion of tubing. The fluid pathway may allow for the flow of fluid from the second portion of tubing through the second connector and the first connector to the first portion of tubing.

In some embodiments, the first connector and second connector provide one way fluid flow. For example, when the first connector is coupled to the second connector, fluid may flow from the second connector to the first connector and not from the first connector to the second connector. In some embodiments, decoupling of the first connector from the second connector results in the flow of fluid from the second connector to the first connector ceasing, thereby preventing leakage when the first connector is decoupled from the second connector. In some embodiments, upon decoupling of the first connector from the second connector, the first connector is sterilized (e.g., via a sterilized cloth or a sterilizing device) or replaced with a new sterile connector to prevent infection or contamination that can occur if the first connector is re-used without sterilization. In some embodiments, the first connector is configured to decouple based on a force that exceeds a predetermined threshold force. When a force is applied to the first connector, such as a pullout force, that exceeds the predetermined threshold force, the first connector may decouple from the second connector. The pullout force may be a force that occurs along the longitudinal axis of the first connector. In some embodiments, the pullout force is caused by tugging or pulling on the first portion of tubing coupled to the first connector. Alternatively, the pullout out force applied to the first connector may be caused by tugging or pulling on the second connector and/or the second portion of tubing coupled to the second connector.

In some embodiments, once the first connector is decoupled from the second connector, the first connector is configured to be re-coupled to the second connector. For example, once the first connector decouples from the second connector (e.g., due to a disconnection event), the first connector may be configured to allow for re-coupling to the second connector after a disconnection event.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to the connection of medical fittings for the administration of medical fluid using the disclosed coupler, it is to be understood that this description is only an example of usage and does not limit the scope of the claims. Various aspects of the disclosed coupler may be used in any application where it is desirable to secure the connection of various tubing and fittings.

The disclosed coupler assembly overcomes several challenges discovered with respect to certain conventional couplers. One challenge with certain conventional couplers is that certain conventional couplers may be improperly secured. Further, during use, certain conventional couplers may be designed to release or dislodge in response to relatively low pullout forces. For example, certain conventional couplers may release in response to pullout forces experienced during patients rolling over in bed, patients catching tubing or lines on bed rails, moving patients to a different bed, fidgeting by pediatric patients, and/or disoriented adult patients pulling out their lines. Indeed, the Association for Vascular Access (AVA) Annual Scientific Meeting in 2017 reported a 10% dislodgement rate for 1,000 patients fitted with peripheral IV catheters, translating to approximately 33 million dislodgements per year in the U.S. alone. Because the accidental or unintentional dislodgement of tubing, catheters, or fittings may interrupt the administration of medical fluids, the use of certain conventional couplers is undesirable.

Therefore, in accordance with the present disclosure, it is advantageous to provide couplers and coupler/connector assemblies as described herein that allows for improved securement of fittings or connectors. The disclosed couplers and coupler/connector assemblies are structured as described herein so as to permit the secure retention of the first connectors, while allowing for decoupling after a disconnection event.

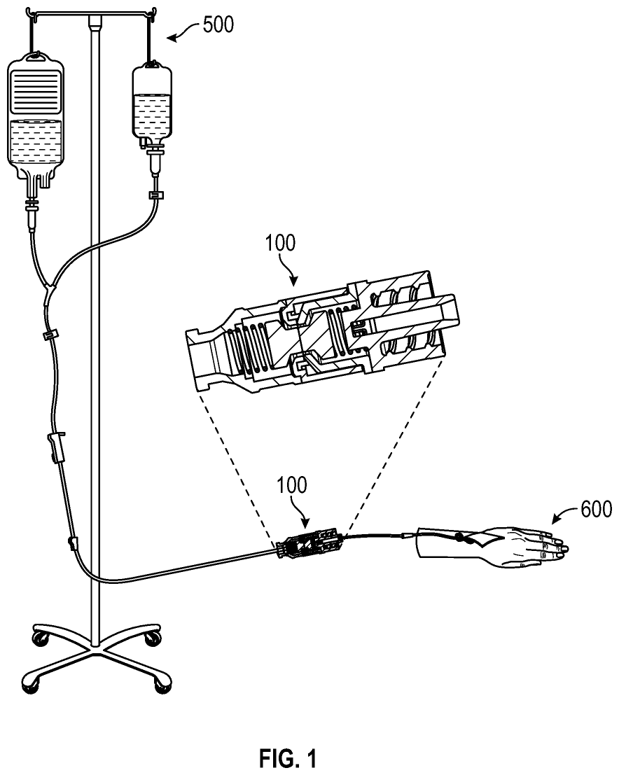

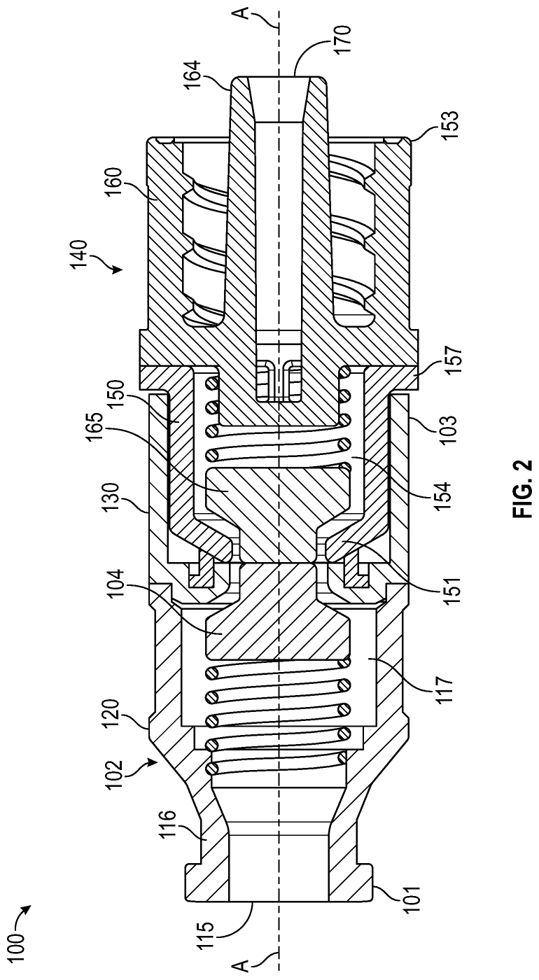

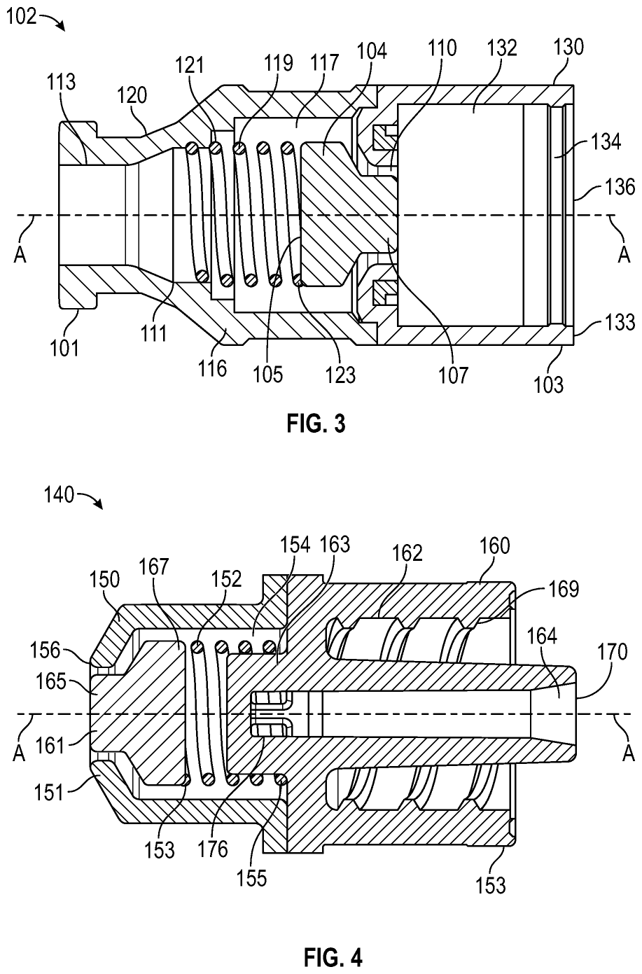

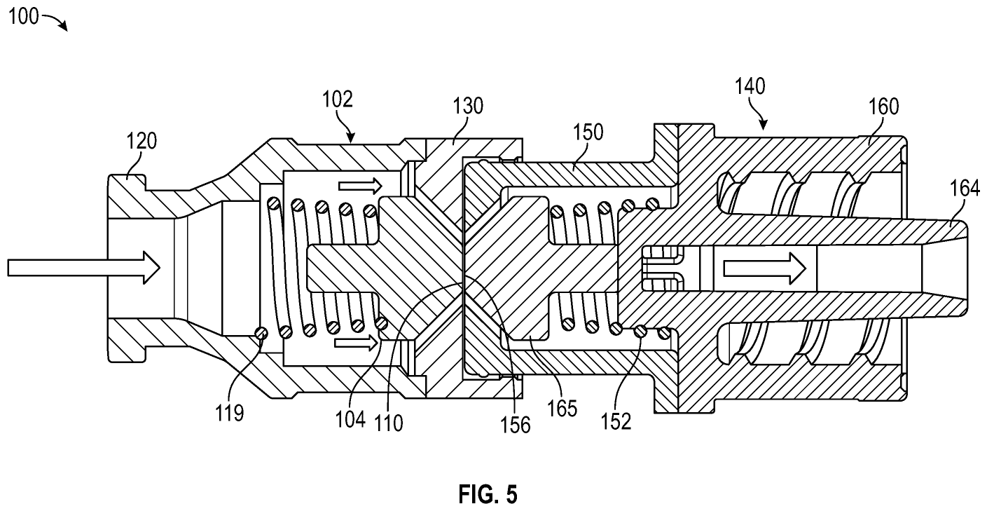

FIG. 1 is a system diagram showing a coupler assembly in use, in accordance with various aspects of the present disclosure. FIG. 2 is a cross-sectional side view of the coupler assembly of FIG. 1, in accordance with various aspects of the present disclosure. FIG. 3 is a cross-sectional side view of a first connector of the coupler assembly of FIG. 1, in accordance with various aspects of the present disclosure. FIG. 4 is a cross-sectional side view of a second connector of the coupler assembly of FIG. 1, in accordance with various aspects of the present disclosure. FIG. 5 is a cross-sectional side view of the coupler assembly of FIG. 1 with the first connector coupled to the second connector, in accordance with various aspects of the present disclosure.

With reference to FIGS. 1-5, coupler assembly 100 allows the flow of a fluid, such as a medical fluid, from fluid source 500 to patient end 600 by releasably coupling a portion of tubing or line with another portion of tubing or line in fluid communication. Coupler assembly 100 may include first connector 102 and second connector 140. First connector 102 may be configured to couple to second connector 140. In some embodiments, first connector 102 and/or second connector 140 are one-way connectors. In the depicted example, portions of tubing can be terminated with connectors/valves, such as first connector 102 and/or second connector 140. In some embodiments, fluid from fluid source 500 flows through coupler assembly 100 to patient end 600. A cannula or needle may be inserted within a patient at patient end 600 allowing medical fluid to flow from fluid source 500 through coupler assembly 100 and into a patient at patient end 600. In some embodiments, decoupling of first connector 102 from second connector 140 interrupts or prevents flow from fluid source 500 to patient end 600.

In some embodiments, coupler assembly 100 includes central axis A-A and first connector 102 and second connector 140 are coupled in series along central axis A-A. First connector 102 and/or second connector 140 may allow for the connection and/or disconnection of tubing to allow for selective fluid communication therebetween. Central axis A-A may extend longitudinally along the length of first connector 102 and second connector 140.

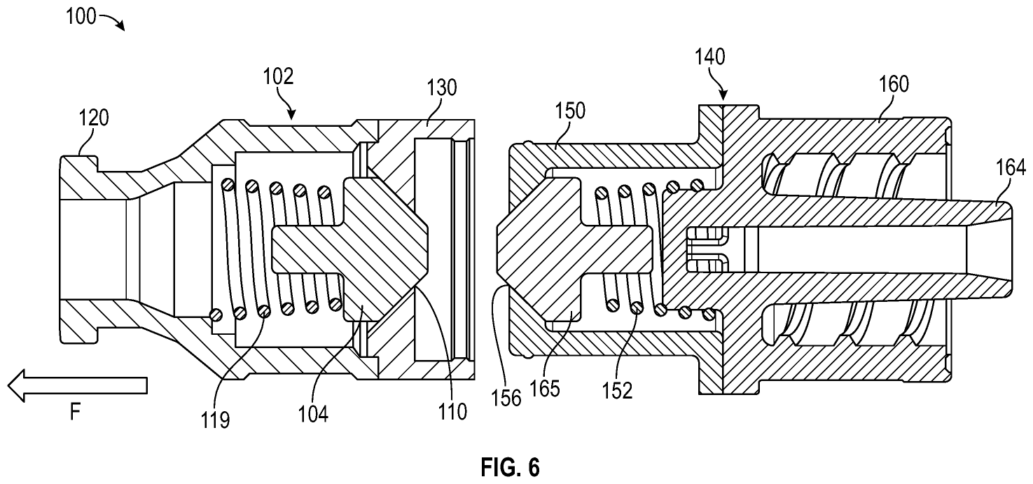

Coupler assembly 100 may have a first configuration (FIG. 2) and a second configuration (FIG. 6). In the first configuration, first connector 102 is coupled to second connector 140. In the second configuration, first connector 102 is decoupled from second connector 140. In some embodiments, coupler assembly 100 transitions from the first configuration to the second configuration in response to a disconnection event. A disconnection even may occur when a pullout force is applied to first connector 102 causing axial movement of first connector 102 relative to second connector 140. In some embodiments, axial movement of first connector 102 relative to second connector 140 is caused when the pullout force applied to first connector 102 exceeds a predetermined threshold force.

In some embodiments, first connector 102 is coupled to a first portion of tubing to allow the first portion of tubing to be connected and/or disconnected with second connector 140. First connector 102 may include first end 101 and second end 103. First end 101 may be coupled to tubing (e.g., a first portion of tubing) and second end 103 may be configured to couple to second connector 140. In some embodiments, a portion of tubing can be coupled with, or engage with first end 101 of first connector 102. First connector 102 via first end 101 may be in fluid communication with the tubing to allow fluid to pass through first connector 102. In some embodiments, first end 101 can have a flat surface to allow for clinicians to easily clean and disinfect first end 101. First end 101 may be in fluid connection with second end 103. First end 101 and second end 103 may be disposed along the longitudinal length of first connector 102. For example, first end 101 and second 103 may be disposed along central axis A-A. First end 101 and/or second end 103 may include an opening to allow first end 101 and/or second end 103 to be in fluid communication with one or more elements (e.g., tubing, connectors, valves, collars, attachments, etc.). For example, first end 101 may be coupled to a tube and second end 103 may include channel 134 to allow for fluid communication through first connector 102. Channel 134 may be configured to receive a portion of second connector 140 (e.g., coupling portion 150) to secure second connector 140 to first connector 102. In some embodiments, channel 134 includes distal or outer edge 133. Edge 133 may be disposed proximate second end 103. In some embodiments, when first connector 102 is coupled to second connector 140, a portion of second connector 140 (e.g., coupling portion 150) extends through second end 103.

In some embodiments, first connector 102 is configured to couple to second connector 140 such that fluid flows into first connector 102 and out of second connector 140. For example, first connector 102 may include an inlet (e.g., inlet 115) configured to receive fluid and second connector 140 may include an outlet (e.g., outlet 170) configured to allow fluid to exit second connector 140. In some embodiments, first connector 102 being coupled to second connector 140 results in the inlet (e.g., inlet 115) of first connector 102 being in fluid communication with the outlet (e.g., outlet 170) of second connector 140.

In some embodiments, fluid can exit or flow through first connector 102 via second end 103 disposed opposite to first end 101. The flow path through first connector 102 can have a straight fluid pathway to make flushing easier and to reduce the risk of hemolysis. Optionally, first connector 102 can include features (e.g., raised features, gripping features) disposed on the outer surface of first connector 102 to allow a clinician to more easily handle or manipulate first connector 102. Some embodiments of first connector 102 may provide connectors that are compatible with connectors of other portions of fluid delivery systems. First connector 102 may be substantially cylindrically shaped.

In some embodiments, first connector 102 includes inlet portion 120 disposed proximate first end 101 and mating portion 130 disposed proximate second end 103. Inlet portion 120 may be configured to couple to a portion of tubing allowing first connector 102 to be in fluid communication with the portion of tubing. For example, inlet portion 120 may include inlet 115 configured to allow for fluid to enter inlet portion 120. Inlet portion 120 may also include channel 113 to allow for the flow of fluid within inlet portion 120. Channel 113 may be disposed within inlet portion 120 and extend from first end 101 to body 116 of inlet portion 120.

In some embodiments, mating portion 130 is disposed opposite inlet portion 120 and is configured to couple to second connector 140 to secure first connector 102 to second connector 140. For exampling mating portion 130 may include one or more grooves 136 configured to receive a portion of second connector 140 to secure and couple first connector 102 to second connector 140, as described below. Groove 109 may be disposed on mating portion 130 proximate second end 103. In some embodiments, groove 136 is configured to receive and engage with a portion of second connector 140 to secure first connector 102 to second connector 140.

Referring to FIGS. 2-3, inlet portion 120 may be coupled to mating portion 130 between first end 101 and second end 103. Inlet portion 120 may be disposed proximate first end 101 and may include channel 113 and interior space 117. Channel 113 may lead into interior space 117. For example, channel 113 may be in fluid communication with interior space 117. In some embodiments, a portion of a tube couples to inlet portion 120 such that the portion of the tube is in fluid communication with channel 113. The portion of the tube being in fluid communication with channel 113 also results in the portion of the tube being in fluid communication with interior space 117.

In some embodiments, inlet portion 120 includes body 116 coupled to channel 113.

Body 116 may include interior space 117. Channel 113 may have a diameter less than an inner diameter of body 116. In some embodiments, the volume of channel 113 is less than the volume of interior space 117 of body 116. Channel 113 may be in fluid communication with interior space 117 of body 116 such that body 116 (e.g., interior space 117) is configured to hold a larger volume of liquid than channel 113.

Mating portion 130 may be disposed proximate second end 103 and may include opening 110, interior space 132, and channel 134. Opening 110 may be in fluid communication with interior space 132 and channel 134. In some embodiments, opening 110 is in fluid communication with interior space 117 of inlet portion 120. For example, opening 110 may be in fluid communication with interior space 117 such that fluid flows from channel 113 into interior space 117 and into opening 110. Fluid may then flow from opening 110 through interior space 132 and exit first connector 102 via channel 134. In some embodiments, channel 134 allows for the flow of fluid out of first connector 102 and into second connector 140 when first connector 102 is coupled to second connector 140. Opening 110 may have a diameter less than channel 134. In some embodiments, channel 134 has a diameter substantially the same as body 116. In some embodiments, first connector 102 is formed by coupling inlet portion 120 to mating portion 130. Alternatively, first connector 102 is formed from a unitary piece.

Referring to FIG. 3, first connector 102 may include valve 104. Valve 104 may be disposed within interior space 117 of body 116. In some embodiments, valve 104 extends, at least partially, into mating portion 130. For example, valve 104 may extend from inlet portion 120 (e.g., interior space 117) into opening 110 of mating portion 130. In some embodiments, valve 104 is configured to seal opening 110 to prevent flow of fluid out of first connector 102 (e.g., leakage). For example, valve 104 may be configured to seal opening 110 to seal off fluid communication between interior space 117 of inlet portion 120 and interior space 132 of mating portion 130. In some embodiments, opening 110 is sized and shaped to match a profile of valve 104 to allow valve 104 to seal opening 110. Valve 104 may have any shape configured to seal opening 110. In some embodiments, first connector 102 allows for one-way flow of fluid. For example, first connector 102 and/or valve 104 may allow for fluid to flow out of opening 110, but not into opening 110.

Valve 104 may have first end 105 and second end 107. Second end 107 may be disposed opposite first end 105. In some embodiments, first end 105 and second end 107 have substantially planar faces or surfaces. The surface of first end 105 may be substantially parallel to the surface of second end 107. In some embodiments, a length and/or width of the surface of second end 107 is less than a length and/or width of the surface of first end 105.

Valve 104 may be coupled to biasing element 119. In some embodiments, valve 104 is coupled to biasing element 119 at first end 105. Biasing element 119 may be a spring or spring-like structure allowing biasing element 119 to be in a compressed state or an expanded state. Biasing element 119 may be disposed within interior space 117. In some embodiments, biasing element 119 is disposed proximate first end 101. Biasing element 119 may be disposed within body 116 such that biasing element 119 is within interior space 117. In some embodiments, biasing element 119 has first end 121 disposed proximate channel 113 and second end 123 disposed proximate valve 104. Valve 104 may be coupled to biasing element at second end 123. For example, second end 123 may be coupled to first end 105 of valve 104. First end 121 may be coupled to distal end 111 of channel 113. For example, channel 113 may turn into interior space 117 at distal end 111 of channel 113. In some embodiments, biasing element 119 is coupled to distal end 111 of channel 113.

Biasing element 119 may have a compressed state and an expanded state. For example, biasing element 119 may be configured to compress (e.g., shorten in length) or expand (e.g., increase in length). Biasing element 119 may be biased to be in the expanded state. In the compressed state, second end 123 is disposed proximate first end 121 and distal end 111 of channel 113. In the expanded state, second end 123 may be disposed away from first end 121. Since valve 104 is coupled to second end 123, in the compressed state valve 104 is disposed proximate first end 121 and distal end 111 compared to when biasing element 119 is in the expanded state. In some embodiments, when biasing element 119 is in the expanded state, valve 104 is disposed away from first end 121 and is at least partially disposed within opening 110. In some embodiments, valve 104 at least partially extends through opening 110 to seal opening 110. Valve 104 being at least partially disposed within opening 110 and extending partially through opening 110 causes opening 110 to be sealed off from interior space 117. For example, biasing element 119 may be in the expanded position resulting in valve 104 being at least partially disposed within opening 110. Valve 104 being disposed within opening 110 prevents inlet portion 120 (e.g., channel 113 and interior space 117) from being in fluid communication with mating portion 130 (e.g., interior space 132 and channel 134).

In some embodiments, biasing element 119 being in expanded state results in valve 104 being in the expanded state and at least partially extending through opening 110. In some embodiments, when valve 104 and biasing element 119 are in the expanded state, second end 107 is disposed through opening 110. For example, when valve 104 and biasing element 119 are in the expanded state, opening 110 may be disposed between first end 105 and second end 107.

In some embodiments, valve 104 is pushed or a pressure/force is applied to valve 104 via another structure to cause biasing element 119 to be in the compressed state resulting in fluid communication between mating portion 130 and inlet portion 120. When the pressure/force is removed from valve 104, biasing element 119 may return to the expanded state resulting in valve 104 being at least partially disposed within opening 110, which causes inlet portion 120 to no longer be in fluid communication with mating portion 130.

Referring to FIGS. 1 and 4, first connector 102 may be configured to couple to second connector 140. Second connector 140 may including coupling portion 150 and output portion 160. Coupling portion 150 may be configured to couple to output portion 160. In some embodiments, coupling portion 150 and output portion 160 form a unitary structure, thereby forming second connector 140.

In some embodiments, second connector 140 is coupled to a second portion of tubing to allow the second portion of tubing to be connected and/or disconnected with first connector 102. Second connector 140 may include first end 151 and second end 153. First end 151 may be configured to couple to first connector 102 and second end 153 may be coupled to tubing (e.g., a second portion of tubing). In some embodiments, a portion of tubing can be coupled with, or engage with second end 153 of second connector 140. Second connector 140 via second end 153 may be in fluid communication with the tubing to allow fluid to pass through second connector 140. In some embodiments, first end 151 can have a flat surface to allow for clinicians to easily clean and disinfect first end 151. First end 151 may be in fluid connection with second end 153. First end 151 and second end 153 may be disposed along the longitudinal length of second connector 140. For example, first end 151 and second end 153 may be disposed along central axis A-A. First end 151 and/or second end 153 may include an opening to allow first end 151 and/or second end 153 to be in fluid communication with one or more elements (e.g., tubing, connectors, valves, collars, attachments, etc.). For example, first end 151 may include opening 156 and second end 153 may be coupled to a tube to allow for fluid communication through second connector 140.

In some embodiments, fluid can exit or flow through second connector 140 via second end 153 disposed opposite to first end 151. The flow path through second connector 140 can have a straight fluid pathway to make flushing easier and to reduce the risk of hemolysis. Optionally, second connector 140 can include features (e.g., raised features, gripping features) disposed on the outer surface of second connector 140 to allow a clinician to more easily handle or manipulate second connector 140. Some embodiments of second connector 140 may provide connectors that are compatible with connectors of other portions of fluid delivery systems. Second connector 140 may be substantially cylindrically shaped.

In some embodiments, second connector 140 includes coupling portion 150 disposed proximate first end 151 and output portion 160 disposed proximate second end 153. Output portion 160 may be configured to couple to a portion of tubing allowing second connector 140 to be in fluid communication with the portion of tubing and to a patient via the portion of tubing. For example, output portion 160 may include channel 164 to allow for the flow of fluid within output portion 160. Channel 164 may be disposed within output portion 160 and extend the length of output portion 160. Channel 164 may include outlet 170. Outlet 170 may be disposed at a distal end of channel 164 and may be disposed proximate second end 153.

In some embodiments, coupling portion 150 is disposed opposite output portion 160 and is configured to couple to first connector 102 to secure first connector 102 to second connector 140. For example, coupling portion 150 may be configured to couple with a portion of first connector 102 to secure and couple first connector 102 to second connector 140, as described below. In some embodiments, coupling portion 150 is sized and shaped to be received by and disposed within first connector 102, such as within mating portion 130. For example, mating portion 130 may be configured to receive and secure coupling portion 150 to secure second connector 140 to first connector 102. In some embodiments, mating portion 130 receives coupling portion 150 such that coupling portion 150 extends through second end 103

In some embodiments, coupling portion 150 includes channel 164 and tube 162. Channel 164 may extend through tube 162. Channel 164 may include proximal end 163, which may include opening 176. Channel 164 may include outlet 170, which may be disposed opposite opening 176 and proximal end 163. Channel 164 may allow outlet 170 to be in fluid communication with opening 176 such that fluid that enters channel 164 at opening 176 (e.g., from first connector 102) flows through channel 164 and out of outlet 170. Tube 162 may be configured to receive and couple to a portion of tubing (e.g., a second portion of tubing). In some embodiments, tube 162 includes grooves 169 disposed on an interior surface of tube 162. Grooves 169 may be configured to assist in securing a portion of tubing within tube 162. For example, a portion of tubing may be inserted into tube 162 and grooves 169 may be configured to assist in preventing the portion of tubing from being inadvertently removed or decoupled from second connector 140.

Coupling portion 150 may be disposed proximate first end 151 compared to output portion 160. Coupling portion 150 may include opening 156. In some embodiments, opening 156 is disposed proximate first end 151. Coupling portion 150 may include interior space 154 disposed within coupling portion 150. In some embodiments, coupling portion 150 includes biasing element 152 and valve 165 disposed within interior space 154. In some embodiments, valve 165 is disposed proximate first end 151 compared to biasing element 152. Biasing element 152 may be a spring coupled to an end of valve 165. Biasing element 152 may be a spring or spring-like structure allowing biasing element 152 to be in a compressed state or an expanded state.

Biasing element 152 may have first end 153 and second end 155. First end 153 of biasing element 152 may be disposed proximate first end 151 compared to second end 155. Biasing element 152 may be coupled to valve 165. In some embodiments, valve 165 is coupled to first end 153 of biasing element 152. Valve 165 may have first end 161 and second end 167. Second end 167 may be disposed opposite first end 161. In some embodiments, first end 161 and second end 167 have substantially planar faces or surfaces. The surface of first end 161 may be substantially parallel to the surface of second end 167. In some embodiments, a length and/or width of the surface of first end 161 is less than a length and/or width of the surface of second end 167. In some embodiments, valve 165 is coupled to biasing element 152 at second end 167.

In some embodiments, biasing element 152 being in expanded state results in valve 165 being in the expanded state and at least partially extending through opening 156. In some embodiments, when valve 165 and biasing element 152 are in the expanded state, first end 161 is disposed through opening 156. For example, when valve 165 and biasing element 152 are in the expanded state, opening 156 may be disposed between first end 161 and second end 167.

In some embodiments, channel 164 extends through output portion 160 into coupling portion 150. For example, channel 164 may extend from an area proximate second end 153 into interior space 154. In some embodiments, opening 176 is disposed within interior space 154. Second end 155 of biasing element 152 may be coupled to channel 164 such that opening 176 is disposed proximate second end 155.

Biasing element 152 may have a compressed state and an expanded state. For example, biasing element 152 may be configured to compress (e.g., shorten in length) or expand (e.g., increase in length). Biasing element 152 may be biased to be in the expanded state. Biasing element 152 may be substantially similar to biasing element 119. In some embodiments, biasing element 152 and biasing element 119 are the same. Alternatively, biasing element 119 and biasing element 152 may have different lengths, different stiffnesses, or may be made out of different materials.

In the compressed state, first end 153 is disposed proximate second end 155 and opening 176 of channel 164. In the expanded state, first end 153 may be disposed away from second end 155. Since valve 165 is coupled to first end 153, in the compressed state valve 165 is disposed proximate second end 155 and opening 176 compared to when biasing element 152 is in the expanded state. In some embodiments, when biasing element 152 is in the expanded state, valve 165 is disposed away from second end 155 and is at least partially disposed within opening 156 and/or through opening 156. Valve 165 being at least partially disposed within opening 156 causes opening 156 to be sealed thereby preventing any fluid from flowing in or out of opening 156 (e.g., leakage). For example, biasing element 152 may be in the expanded position resulting in valve 165 being at least partially disposed within opening 156. Valve 165 being disposed within and/or through opening 156 seals opening 156 and prevents liquid from entering or exiting second connector 140 (e.g., leakage). In some embodiments, opening 156 is sized and shaped to match a profile of valve 165 to allow valve 165 to seal opening 156. Valve 165 may have any shape configured to seal opening 156. In some embodiments, second connector 140 allows for one-way flow of fluid. For example, second connector 140 and/or valve 165 may allow for fluid to flow into opening 156, but not out of opening 156.

In some embodiments, first end 153 of biasing element 152 is coupled to valve 165. Valve 165 may be disposed within interior space 154 and may be configured to at least partially extend through opening 156. In some embodiments, valve 165 is configured to seal opening 156 when biasing element 152 is in the expanded state. For example, valve 165 may be fully disposed within opening 156 and at least partially extend through opening 156 to seal opening 156.

In some embodiments, valve 165 is pushed or a pressure/force is applied to valve 165 via another structure to cause biasing element 152 to be in the compressed state resulting in fluid being able to flow in and/or out of opening 156. When the pressure/force is removed from valve 165, biasing element 152 may return to the expanded state resulting in valve 165 being at least partially disposed within opening 156, thereby sealing opening 156 and preventing fluid from flowing in and/or out of opening 156 (e.g., leakage).

In some embodiments, when biasing element 152 is in the compressed state and valve 165 is not sealing opening 156 and opening 156 is in fluid communication with channel 164. For example, fluid may flow into opening 156 and into interior space 154. The fluid may flow from interior space 154 into channel 164 via opening 176. Fluid may then flow through channel 164 into a tubing portion coupled to output portion 160 via tube 162 and grooves 169. Biasing element 152 being in the expanded position results in valve 165 sealing opening 156 and opening 156 no longer being in fluid communication with channel 164.

Referring to FIG. 2, first connector 102 may be configured to couple to second connector 140. In some embodiments, coupling first connector 102 to second connector 140 results in inlet 115 being in fluid communication with outlet 170. For example, coupling first connector 102 to second connector 140 results in interior space 117 being in fluid communication with interior space 154. In some embodiments, coupling first connector 102 and second connector 140 results in mating portion 130 receiving a majority or at least a portion of coupling portion 150. For example, mating portion 130 may include channel 134 configured to receive and secure coupling portion 150. Mating portion 130 may include one or more grooves 136 configured to secure coupling portion 150 within mating portion 130.

Mating portion 130 may be configured to secure second connector 140 to first connector 102 such that second connector 140 is prevented from inadvertently being decoupled from first connector 102. For example, mating portion 130 may be configured to be secured second connector 140 by friction fitting coupling portion 150 within mating portion 130. In some embodiments, second connector 140 is coupled and secured to first connector 102 by friction fitting or snap fitting coupling portion 150 within mating portion 130. Coupling portion 150 may be disposed and secured within mating portion 130 via snap fitting, friction fitting, magnets, male and female threadings, adhesives, or any other type of securing element. For example, coupling portion 150 may be disposed and secured within mating portion 130 by snap fitting, friction fitting, or threadably engaging coupling portion 150 to mating portion 130.

Grooves 136 may assist in securing coupling portion 150 within mating portion 130. In some embodiments, coupling portion 150 may be inserted through channel 134 such that opening 156 of second connector 140 is disposed proximate opening 110 of first connector 102. In some embodiments, when first connector 102 is coupled to second connector 140, opening 110 contacts or abuts opening 156.

Coupling portion 150 may include ring 157 configured to prevent second connector 140 from being inserted more into mating portion 130. For example, ring 157 may be a protruding ring around coupling portion 150 that may about an outer edge (e.g., 133) of channel 134 when first connector 102 is coupled to second connector 140. Ring 157 may prevent second connector 140 from being inserted through opening 110 of first connector and/or may prevent first connector 102 from being inserted through opening 156 of second connector.

When first connector 102 is not coupled to second connector 140, valve 104 may be extend into and through opening 110 and valve 165 may extend into and through opening 156. IN some embodiments, valve 104 and valve 165 may both be in an expanded state when biasing element 119 and biasing element 152, respectively, are in the expanded state. Valve 104 being in the expanded results in valve 104 sealing opening 110 by extending into and through opening 110. Valve 165 being in the expanded results in valve 165 sealing opening 156 by extending into and through opening 156. Biasing element 119 and biasing element 152 being in compressed state results in valve 104 and valve 165, respectively, being in the compressed state. When valve 104 is in the compressed state, valve 104 no longer seals opening 110, and when valve 165 is in the compressed state, valve 165 no longer seals opening 156. In some embodiments, when first connector 102 is decoupled from second connector 140 (e.g., coupler assembly 100 being in the second configuration), valve 104 seals opening 110 and valve 165 seals opening 156 resulting in no flow of fluid out of opening 110 and opening 156. Valve 104 (and biasing element 119) and valve 165 (and biasing element 152) may transition from the expanded state to compressed state due to a force being applied to each of valve 104 and valve 165.

In some embodiments, when first connector 102 is coupled to second connector 140 (e.g., coupler assembly 100 being in first configuration), valve 104 applies a force on valve 165 resulting in valve 104 (and biasing element 119) being in the compressed state, and valve 165 applies a force on valve 104 resulting in valve 165 (and biasing element 152) being in the compressed state. In other words, when first connector 102 is coupled to second connector 140, valve 104 and valve 165 apply a force on each other resulting in both valve 104 and valve 165 being in the compressed state and allowing opening 110 to be in fluid communication with opening 156.

In some embodiments, coupling first connector 102 to second connector 140 results in opening 110 abutting or being disposed proximate opening 156. Due to valve 104 extending through opening 110 and valve 165 extending through opening 156, when opening 110 is abutting or disposed proximate opening 156, such as when first connector 102 is coupled to second connector 140, valve 104 and valve 165 may exert forces (e.g., pressure) on each other resulting in valve 104 and valve 165 being in the compressed state. Valve 104 and valve 165 being in the compressed state results in opening 110 and opening 156 no longer be sealed and thus allows for fluid to flow out of opening 110 and into opening 156.

In practice, second connector 140 is coupled to first connector 102 by inserting coupling portion 150 into mating portion 130. Coupling portion 150 may be inserted into mating portion 130 until opening 156 abuts opening 110 and/or until an outer edge (e.g., edge 133) of channel 134 abuts ring 157. Opening 156 abutting opening 110 results in valve 104 contacting valve 165 since valve 104 extends at least partially through opening 110 and valve 165 extends at least partially through opening 156. Valve 104 contact valve 165 results in each applying a force or pressure to one another. Due to valve 104 being coupled to biasing element 119 and valve 165 being coupled to biasing element 152, upon application of the force or pressure, each of valve 104 and valve 165 may transition from the expanded state to the compressed state resulting in opening 110 and opening 156, respectively, no longer be sealed. Opening 110 may be in fluid communication with opening 156 due to opening 110 abutting opening 156, and opening 110 and opening 156 no longer being sealed by valve 104 and 165, respectively. In some embodiments, opening 110 being in fluid communication with opening 156 results in fluid being able to flow from first connector 102 to second connector 140 through opening 110 and opening 156.

Referring to FIG. 5, opening 110 being in fluid communication opening 156 results in a fluid pathway (denoted by arrows in FIG. 5) being formed between first connector 102 and second connector 140. For example, when first connector 102 is coupled to second connector 140, opening 110 may be in fluid communication with opening 156 resulting in a fluid pathway being formed between first connector 102 and second connector 140. The fluid pathway may be formed when coupler assembly 100 is in the first configuration (e.g., when first connector 102 is coupled to second connector 140). In the first configuration, fluid may flow into first connector 102 via inlet 115. Fluid may then flow within interior space 117 of body 116, around biasing element 119 and valve 104, which may be in the compressed state, and through opening 110. The fluid may then flow from opening 110 through opening 156 and into interior space 154 ad around valve 165 and biasing element 152. The fluid may flow from interior space 154 into opening 176 and through channel 164 out of outlet 170 to a portion of tubing coupled to output portion 160.

FIG. 6 is a cross-sectional side view of the coupler assembly of FIG. 1 with the first connector decoupled from the second connector, in accordance with various aspects of the present disclosure.

Referring to FIG. 6, coupler assembly 100 may be configured to be in a second configuration. In the second configuration, first connector 102 is decoupled from second connector 140. In the second configuration, biasing element 119 and valve 104, and valve 165 and biasing element 152 return to their biased expanded state and the fluid pathway between first connector 102 and second connector 140 is interrupted. In some embodiments, biasing element 119 and valve 104 returning to the expanded state results in opening 110 being sealed and prevent flow of fluid in/out of first connector 102. In some embodiments, biasing element 152 and valve 165 returning to the expanded state results in opening 156 being sealed by valve 165, preventing flow of fluid in/out of second connector 140. Coupler assembly 100 may transition from the first configuration to the second configuration by first connector 102 decoupling from second connector 140.

In some embodiments, first connector 102 is configured to decouple from second connector 140 due to a disconnection event, which is caused by a pullout force. For example, a pullout force (e.g., force F) may be applied to first connector 102, either by being directly applied to first connector 102 or indirectly applied to first connector 102, such as being applied to tubing coupled to first connector 102. The pullout force may cause first connector 102 to move axially away from and second connector 140 along central axis A-A thereby decoupling first connector 102 second connector 140.

In some embodiments, first connector 102 is decoupled second connector 140 when force F exceeds a predetermined threshold force. For example, if force F is less than the predetermined threshold force, first connector 102 may not decouple from second connector 140. The predetermined threshold force prevents inadvertent or accidental decoupling based on minor forces or movements. The predetermined threshold force may be based on the flexibility and/or stiffness of mating portion 130 and/or grooves 136. For example, the higher the stiffness of mating portion 130 and/or grooves 136, the higher the predetermined threshold force. In some embodiments, mating portion 130 includes grooves 136 disposed at different positions within channel 134 to increase the frictional force on coupling portion 150 to prevent inadvertent decoupling of coupling portion 150 (and second connector 140) from first connector 102. For example, multiple grooves 136 results in multiple positions or snap positions for securing coupling portion 150 within mating portion 130. The multiple positions allow for different forces required to decouple second connector 140 from first connector 102. The multiple positions and different forces may be required based on the desired use of coupler assembly 100. In some embodiments, when the pullout force exceeds the predetermined threshold force, mating portion 130 and/or grooves 136 may fail to retain coupling portion 150 within mating portion 130 thereby permitting decoupling of the first connector 102 from second connector 140.

In some embodiments, the predetermined threshold force is approximately 4 pounds (lbs). The predetermined threshold force may be from approximately 1 lb to approximately 8 lbs, approximately 3 lbs to approximately 7 lbs, approximately 4 lbs to approximately 6 lbs, or greater than 8 lbs. For example, a patient may have a needle/catheter inserted into their skin and the needle/catheter may be coupled to first connector 102 or second connector 140. The patient may walk away from an infusion pump or accidental pull on a fluid line coupled to first connector 102 or second connector 140 and the force exceeds 4 lbs, first connector 102 may automatically release or decouple from second connector 140, effectively closing the fluid pathway between first connector 102 and second connector 140, as described herein.

In some embodiments, upon decoupling of first connector 102 from second connector 140, a user sterilizes first connector 102 and recouples first connector 102 to second connector 140 by inserting coupling portion 150 back into mating portion 130. In some embodiments, a user may sterilize first connector 102 and/or second connector 140. Recoupling first connector 102 to second connector 140 results in coupler assembly 100 transitioning from the second configuration to the first configuration.

The disclosures described herein include at least the following clauses:

Clause 1: a coupler comprising a first connector having a first end, a second end opposite the first end, a first opening, and a first valve disposed between the first end and the second end, the first valve having a compressed state and an expanded state, the first valve extending at least partially through the first opening when the first valve is in the expanded state; and a second connector having a coupling portion, a second opening, and a second valve disposed at least partially within the coupling portion, the second valve having a compressed state and an expanded state, the second valve extending at least partially through the second opening when the second valve is in the expanded state. The first connector is configured to receive at least a portion of the second connector to detachably couple the first connector to the second connector such that the coupling portion of the second connector extends through the second end of the first connector, and the first valve and the second valve are in the compressed state when the first connector is coupled to the second connector such that a fluid pathway is formed through the first connector and the second connector. The first connector is configured to decouple from the second connector in response to a pullout force exceeding a predetermined threshold force.

Clause 2: The coupler of clause 1, wherein the first valve seals the first opening when the first valve is in the expanded state.

Clause 3: The coupler of clause 1, wherein the second valve seals the second opening when the second valve is in the expanded state.

Clause 4: The coupler of clause 1, wherein the first connector includes a first biasing element coupled to the first valve, the first biasing element having an expanded state and a compressed state.

Clause 5: The coupler of clause 4, wherein the first biasing element being in the expanded state results in the first valve being in the expanded state and the first biasing element being in the compressed state results in the first valve being in the compressed state.

Clause 6: The coupler of clause 1, wherein the second connector includes a second biasing element coupled to the second valve, the second biasing element having an expanded state and a compressed state.

Clause 7: The coupler of clause 6, wherein the second biasing element being in the expanded state results in the second valve being in the expanded state and the second biasing element being in the compressed state results in the second valve being in the compressed state.

Clause 8: The coupler of clause 1, wherein the pullout force is a force applied to the first connector along a central axis of the first connector and the central axis extends at least along a length of the first connector.

Clause 9: The coupler of clause 8, wherein the central axis extends through the first connector and the second connector when the first connector is coupled to the second connector.

Clause 10: The coupler of clause 1, wherein the first valve includes a first substantially planar surface, and the second valve includes a second substantially planar surface, the first substantially planar surface configured to contact the second substantially planar surface when the first connector is coupled to the second connector.

Clause 11: The coupler of clause 1, wherein the first valve exerts a pressure on the second valve causing the second valve to be in the compressed state when the first connector is coupled to the second connector.

Clause 12: The coupler of clause 1, wherein the first valve at least partially extends through the first opening to seal the first opening when the first connector is decoupled from the second connector.

Clause 13: The coupler of clause 1, wherein the second valve at least partially extends through the second opening to seal the second opening when the first connector is decoupled from the second connector.

Clause 14: The coupler of clause 1, wherein the first connector includes a mating portion configured to receive the coupling portion of the second connector when the first connector is coupled to the second connector.

Clause 15: The coupler of clause 1, wherein the second valve is in the expanded state when the first connector is disconnected from the second connector.

Clause 16: The coupler of clause 1, the first valve is in the expanded state when the first connector is disconnected from the second connector.

Clause 17: The coupler of clause 1, wherein the first connector is configured to remain coupled to the second connector when the pullout force does not exceed the predetermined threshold force.

Clause 18: The coupler of clause 1, wherein the first valve does not overlap with the second valve.

Clause 19: The coupler of clause 1, wherein the first valve abuts the second valve when the first connector is coupled to second connector.

Clause 20: The coupler of clause 1, wherein the coupler has a first configuration and in the first configuration the first connector is coupled to the second connector such that the second connector is at least partially disposed within the first connector.

Clause 21: The coupler of clause 1, wherein the coupler has a second configuration and in the second configuration the first connector is disconnected from the second connector.

Clause 22: The coupler of clause 1, wherein the first connector is coupled to a first portion of tubing at the first end and the second connector is coupled to a second portion of tubing at an output portion.

Clause 23: A coupler comprising a first connector having a first end, a second end opposite the first end, a mating portion disposed proximate the second end, a first opening disposed between the first end and the second end, a biasing element disposed within the first connector, and a first valve coupled to the biasing element and disposed between the first end and the first opening, the first valve and the first biasing element having a compressed state and an expanded state, the first biasing element being in the expanded state causes the first valve to be in the expanded state resulting in the first valve at least partially extending through the first opening to seal the first opening, and a second connector having a coupling portion, a biasing element disposed within the second connector, a second opening, and a second valve coupled to the biasing element and disposed at least partially within the coupling portion, the second valve and the second biasing element having a compressed state and an expanded state, the second biasing element being in the expanded state causes the second valve to be in the expanded state resulting in the second valve at least partially extending through the second opening to seal the second opening. the mating portion of first connector is configured to receive the coupling portion of the second connector to detachably couple the first connector to the second connector such that the coupling portion of the second connector extends through the second end of the first connector, and the first biasing element being in the compressed state causes the first valve to be in the compressed state and the second biasing element being in the compressed state causes the second valve to be in the compressed state such that a fluid pathway is formed through the first connector and the second connector via the first opening and the second opening. The first connector is configured to decouple from the second connector in response to a pullout force exceeding a predetermined threshold force.

Clause 24: A coupler comprising a first connector having a first end, a second end opposite the first end, an inlet portion disposed proximate the first end, a mating portion in fluid communication with the inlet portion and disposed proximate the second end, a first opening disposed between the first end and the second end, a first interior space disposed between the first end and the first opening, a biasing element disposed within the interior space, and a first valve coupled to the biasing element and disposed between the first end and the first opening, the first valve and the first biasing element having a compressed state and an expanded state, the first biasing element being in the expanded state causes the first valve to be in the expanded state resulting in the first valve at least partially extending through the first opening to seal the first opening, and a second connector having a coupling portion, an output portion extending from the coupling portion, an interior space disposed within the coupling portion, a biasing element disposed within the interior space, a second opening, and a second valve coupled to the biasing element and disposed at least partially within the coupling portion, the second valve and the second biasing element having a compressed state and an expanded state, the second biasing element being in the expanded state causes the second valve to be in the expanded state resulting in the second valve at least partially extending through the second opening to seal the second opening. The mating portion of first connector is configured to receive the coupling portion of the second connector to detachably couple the first connector to the second connector such that the coupling portion of the second connector extends through the second end of the first connector, and the first biasing element being in the compressed state causes the first valve to be in the compressed state and the second biasing element being in the compressed state causes the second valve to be in the compressed state such that a fluid pathway is formed through the first connector and the second connector via the first opening and the second opening. The first valve includes a first substantially planar surface, and the second valve includes a second substantially planar surface, the first substantially planar surface configured to contact the second substantially planar surface when the first connector is coupled to the second connector. The first connector is configured to decouple from the second connector in response to a pullout force exceeding a predetermined threshold force, the pullout force being a force applied to the first connector along a central axis of the first connector and the central axis extends at least along a length of the first connector.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples, and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A coupler comprising:
a first connector having a first end, a second end opposite the first end, a first opening, and a first valve disposed between the first end and the second end, the first valve having a compressed state and an expanded state, the first valve extending at least partially through the first opening when the first valve is in the expanded state; and
a second connector having a coupling portion, a second opening, and a second valve disposed at least partially within the coupling portion, the second valve having a compressed state and an expanded state, the second valve extending at least partially through the second opening when the second valve is in the expanded state,
wherein the first connector is configured to receive at least a portion of the second connector to detachably couple the first connector to the second connector such that the coupling portion of the second connector extends through the second end of the first connector, and the first valve and the second valve are in the compressed state when the first connector is coupled to the second connector such that a fluid pathway is formed through the first connector and the second connector,
wherein the first connector is configured to decouple from the second connector in response to a pullout force exceeding a predetermined threshold force.

2. The coupler of claim 1, wherein the first valve seals the first opening when the first valve is in the expanded state.

3. The coupler of claim 1, wherein the second valve seals the second opening when the second valve is in the expanded state.

4. The coupler of claim 1, wherein the first connector includes a first biasing element coupled to the first valve, the first biasing element having an expanded state and a compressed state.

5. The coupler of claim 4, wherein the first biasing element being in the expanded state results in the first valve being in the expanded state and the first biasing element being in the compressed state results in the first valve being in the compressed state.

6. The coupler of claim 1, wherein the second connector includes a second biasing element coupled to the second valve, the second biasing element having an expanded state and a compressed state.

7. The coupler of claim 6, wherein the second biasing element being in the expanded state results in the second valve being in the expanded state and the second biasing element being in the compressed state results in the second valve being in the compressed state.

8. The coupler of claim 1, wherein the pullout force is a force applied to the first connector along a central axis of the first connector and the central axis extends at least along a length of the first connector.

9. The coupler of claim 8, wherein the central axis extends through the first connector and the second connector when the first connector is coupled to the second connector.

10. The coupler of claim 1, wherein the first valve includes a first substantially planar surface and the second valve includes a second substantially planar surface, the first substantially planar surface configured to contact the second substantially planar surface when the first connector is coupled to the second connector.

11. The coupler of claim 1, wherein the first valve exerts a pressure on the second valve causing the second valve to be in the compressed state when the first connector is coupled to the second connector.

12. The coupler of claim 1, wherein the first valve at least partially extends through the first opening to seal the first opening when the first connector is decoupled from the second connector.

13. The coupler of claim 1, wherein the second valve at least partially extends through the second opening to seal the second opening when the first connector is decoupled from the second connector.

14. The coupler of claim 1, wherein the first connector includes a mating portion configured to receive the coupling portion of the second connector when the first connector is coupled to the second connector.

15. The coupler of claim 1, wherein the first valve and the second valve are in the expanded state when the first connector is disconnected from the second connector.

16. The coupler of claim 1, wherein the first connector is configured to remain coupled to the second connector when the pullout force does not exceed the predetermined threshold force.

17. The coupler of claim 1, wherein the coupler has a first configuration and in the first configuration the first connector is coupled to the second connector such that the second connector is at least partially disposed within the first connector.

18. The coupler of claim 1, wherein the first connector is coupled to a first portion of tubing at the first end and the second connector is coupled to a second portion of tubing at an output portion.

19. A coupler comprising:
a first connector having a first end, a second end opposite the first end, a mating portion disposed proximate the second end, a first opening disposed between the first end and the second end, a biasing element disposed within the first connector, and a first valve coupled to the biasing element and disposed between the first end and the first opening, the first valve and the first biasing element having a compressed state and an expanded state, the first biasing element being in the expanded state causes the first valve to be in the expanded state resulting in the first valve at least partially extending through the first opening to seal the first opening; and
a second connector having a coupling portion, a biasing element disposed within the second connector, a second opening, and a second valve coupled to the biasing element and disposed at least partially within the coupling portion, the second valve and the second biasing element having a compressed state and an expanded state, the second biasing element being in the expanded state causes the second valve to be in the expanded state resulting in the second valve at least partially extending through the second opening to seal the second opening,
wherein the mating portion of first connector is configured to receive the coupling portion of the second connector to detachably couple the first connector to the second connector such that the coupling portion of the second connector extends through the second end of the first connector, and the first biasing element being in the compressed state causes the first valve to be in the compressed state and the second biasing element being in the compressed state causes the second valve to be in the compressed state such that a fluid pathway is formed through the first connector and the second connector via the first opening and the second opening,
wherein the first connector is configured to decouple from the second connector in response to a pullout force exceeding a predetermined threshold force.

20. A coupler comprising:
a first connector having a first end, a second end opposite the first end, an inlet portion disposed proximate the first end, a mating portion in fluid communication with the inlet portion and disposed proximate the second end, a first opening disposed between the first end and the second end, a first interior space disposed between the first end and the first opening, a biasing element disposed within the interior space, and a first valve coupled to the biasing element and disposed between the first end and the first opening, the first valve and the first biasing element having a compressed state and an expanded state, the first biasing element being in the expanded state causes the first valve to be in the expanded state resulting in the first valve at least partially extending through the first opening to seal the first opening; and
a second connector having a coupling portion, an output portion extending from the coupling portion, an interior space disposed within the coupling portion, a biasing element disposed within the interior space, a second opening, and a second valve coupled to the biasing element and disposed at least partially within the coupling portion, the second valve and the second biasing element having a compressed state and an expanded state, the second biasing element being in the expanded state causes the second valve to be in the expanded state resulting in the second valve at least partially extending through the second opening to seal the second opening,
wherein the mating portion of first connector is configured to receive the coupling portion of the second connector to detachably couple the first connector to the second connector such that the coupling portion of the second connector extends through the second end of the first connector, and the first biasing element being in the compressed state causes the first valve to be in the compressed state and the second biasing element being in the compressed state causes the second valve to be in the compressed state such that a fluid pathway is formed through the first connector and the second connector via the first opening and the second opening,
wherein the first valve includes a first substantially planar surface, and the second valve includes a second substantially planar surface, the first substantially planar surface configured to contact the second substantially planar surface when the first connector is coupled to the second connector,
wherein the first connector is configured to decouple from the second connector in response to a pullout force exceeding a predetermined threshold force, the pullout force being a force applied to the first connector along a central axis of the first connector and the central axis extends at least along a length of the first connector.
